(19) United States
(12) Plant Patent Application Publication
(10) Pub. No.: US 2016/0278270 P1
(43) Pub. Date: Sep. 22, 2016

(54) Osteospermum plant named 'KLEOE14243'

(50) ;

(71) Applicant: Klemm+Sohn GmbH & Co. KG, Stuttgart (DE)

(72) Inventors: Nils Klemm, Stuttgart (DE); Silke Wagener, Leinfelden-Echterdingen (DE)

(73) Assignee: Klemm+Sohn GmbH & Co. KG, Stuttgart (DE)

(21) Appl. No.: 14/545,028

(22) Filed: Mar. 18, 2015

(51) Int. Cl. A01H 5/00

(52) U.S. Cl. PLT/360

(57) ABSTRACT

A new Osteospermum plant particularly distinguished by yellow ray florets and a semi-upright growth habit, is disclosed.

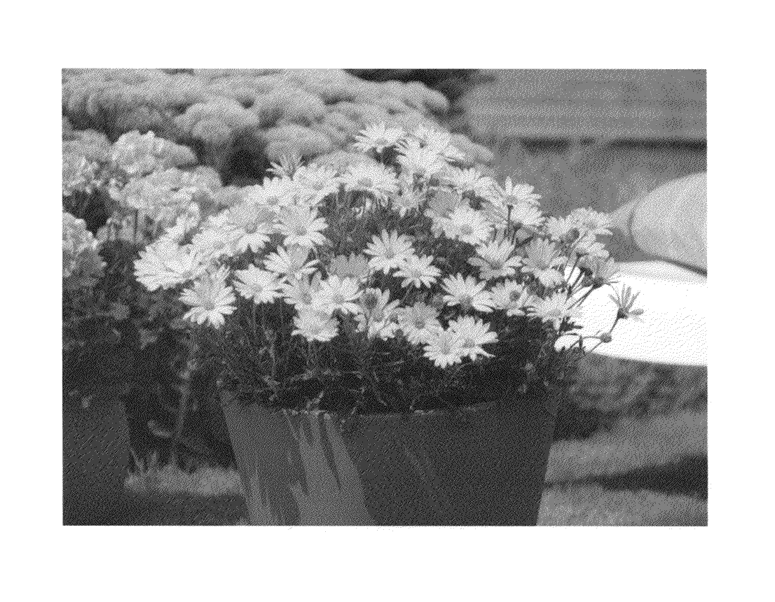

### GENUS AND SPECIES

Osteospermum ecklonis Norl.

### VARIETY DENOMINATION

‘KLEOE14243’

### BACKGROUND OF THE NEW PLANT

The present invention comprises a new and distinct variety of Osteospermum, botanically known as Osteospermum ecklonis, and hereinafter referred to by the variety name ‘KLEOE14243’. ‘KLEOE14243’ originated from a controlled cross conducted in summer 2010 in Nairobi, Kenya between the proprietary female Osteospermum variety ‘OE-2009-0827’ (unpatented), and the proprietary male Osteospermum variety ‘OE-2000-BA01’ (unpatented).

The seeds from the cross were sown and plants were grown in a greenhouse for evaluation, where an individual plant designated ‘KLEOE14243’ was selected from the group of plants in April 2011. In May 2011, ‘KLEOE14243’ was first vegetatively propagated by terminal vegetative cuttings. ‘KLEOE14243’ was found to reproduce true to type in successive generations of asexual propagation via terminal tip vegetative cuttings.

### SUMMARY

The following are the most outstanding and distinguishing characteristics of this new variety when grown under normal horticultural practices in Stuttgart, Germany.

1. Yellow ray florets; and

2. A semi-upright growth habit.

### DESCRIPTION OF THE PHOTOGRAPH

This new Osteospermum plant is illustrated by the accompanying photograph which shows the plant's overall plant habit including form, foliage, and flowers. The photograph is of a four-month-old plant grown outdoors in Stuttgart, Germany in August 2012. The colors shown are as true as can be reasonably obtained by conventional photographic procedures.

### DESCRIPTION OF THE NEW VARIETY

The following detailed descriptions set forth the distinctive characteristics of ‘KLEOE14243’. The data which define these characteristics were collected from asexual reproductions carried out in Stuttgart, Germany. Data was collected on five-month-old plants grown in a greenhouse in Stuttgart, Germany in March 2015 using CCC 0.1% and 0.2% for growth regulation and artificial light, SONT and LED for 16 hours in alternation. Color references are to The R.H.S. Colour Chart of The Royal Horticultural Society of London (R.H.S.), 5^{th} edition (2007).
Classification:
   Family.—Asteraceae.
   Botanical.—Osteospermum ecklonis.
   Common.—Osteospermum; African daisy.
   Designation.—‘KLEOE14243’.
   Parentage.—Female parent: The proprietary female Osteospermum variety ‘OE-2009-0827’ (unpatented) Male parent: The proprietary male Osteospermum variety ‘OE-2000-BA01’ (unpatented).
Plant:
   Form and habit.—Herbaceous perennial but usually cultivated as an annual; not frost resistant.
   Growth habit.—Semi-upright.
   Branching habit.—Freely branching.
   Height (from top of soil, including inflorescence).—About 20.0 cm.
   Width (including inflorescences).—About 30.0 cm.
   Propagation.—Terminal tip cuttings.
   Time to produce a finished flowering plant.—14 weeks (winter to spring).
   Time to initiate and develop roots.—About 3 weeks.
   Root description.—Freely rooting, light tan in color.
Leaves:
   Arrangement.—Alternate.
   Shape.—Oblanceolate.
   Apex.—Acute.
   Base.—Acute.
   Margin.—Medium indentation of margin/serrate.
   Immature color.—Upper surface: RHS 141B Lower surface: RHS 141C.
   Mature color.—Upper surface: RHS 139A Lower surface: RHS 141C.
   Length.—About 5.0 cm to 6.0 cm.
   Width.—About 2.0 cm.
   Texture and appearance (upper and lower surfaces).—Leathery with a covering of fine hairs.
   Petioles.—Absent.
   Venation pattern.—Pinnate.
   Venation color.—Upper surface: RHS 144A Lower surface: RHS 141C.
Stems:
   Total number of branches per plant (calculated at the base of the plant).—About 7.
   Length.—Up to 14.0 cm.
   Diameter.—About 0.6 cm.
   Internode length.—Between 1.0 cm and 2.0 cm.
   Color.—RHS 144B.
   Texture.—Pubescent, a covering of fine hairs.
   Anthocyanin.—Absent.
Flower buds:
   Shape.—Ovate.
   Length.—About 1.0 cm to 1.5 cm.
   Diameter.—About 1.0 cm.
   Color (at tight bud just before the ray florets unfold).—RHS 143B.
Inflorescence:
   Type.—Composite, single.
   Blooming habit.—Spring to autumn.
   Quantity of inflorescences per plant.—About 30.
   Lastingness of the inflorescences on the plant.—About 2 weeks.
   Fragrance.—Absent to faint.
   Inflorescence diameter.—About 7.0 cm.
   Inflorescence depth.—About 1.5 cm.
   Disc diameter.—About 1.5 cm.
Disc floret:
   Quantity per inflorescence.—70 to 80.
   Shape.—Lanceolate.
   Tube color.—Closed: RHS 145A Mature: RHS 1C.
   Length.—About 0.5 cm.
   Diameter (at apex).—About 0.1 cm.
   Apex.—Obtuse.
   Apex color.—RHS 1C.
   Base.—Acute.
   Base color.—RHS 157D.
   Margin.—Entire.
Ray floret:
   Quantity per inflorescence.—About 20.
   Shape.—Oblanceolate.
   Color.—Upper surface: RHS 9A and RHS 6A at the base Lower surface: RHS 12A.
   Length.—3.0 cm to 3.5 cm.
   Width.—0.9 cm.
   Apex.—Obtuse.
   Base.—Acute.
   Margin.—Entire.
   Texture (both upper and lower surfaces).—Smooth.
Peduncle:
   Length.—Up to 8.0 cm.
   Diameter.—About 0.2 cm.
   Texture.—Pubescent, a covering of fine hairs.
   Color.—RHS 144B.
Phyllaries:
   Texture.—Pubescent, a covering of fine hairs.
   Color.—RHS 143A.
   Arrangement.—Single.
   Observed quantity per flower.—About 20.
   Shape.—Lanceolate.
   Color.—Upper surface: RHS 143A Lower surface: RHS 143B.
   Length.—About 1.0 cm.
   Width.—0.1 cm to 0.2 cm.
   Apex.—Acute.
   Base.—Obtuse.
   Margin.—Entire.
   Texture.—Upper surface: Pubescent, a covering of fine hairs Lower surface: Shiny.
Reproductive organs:
   Androecium.—Location: On the disc floret only Stamens: Quantity: Numerous Shape: Filamentous Filament length: About 0.5 cm Filament diameter: Less than 0.005 cm Anther: Shape: Lanceolate Color: RHS 203A Length: About 0.3 cm Diameter: Less than 0.05 cm Pollen: Color: RHS 14A Amount: Moderate.
   Gynoecium.—Location: On the base of the ray florets, reduced on disc florets Pistils: Number: About 20 fully developed on ray florets Length: 0.3 cm Diameter: Less than 0.05 cm Stigma: Color: RHS 6C Shape: Split in two parts at the end Length: About 0.1 cm Diameter: Less than 0.05 cm Style: Color: RHS 4D Length: 0.3.cm Diameter: Less than 0.05 cm.
Fruit and seed set: None observed.
Disease and pest/insect resistance: No disease and pest/insect resistance observed.

### COMPARISON WITH PARENTAL LINES AND KNOWN VARIETY

‘KLEOE14243’ is distinguished from its parents, the female parent ‘OE-2009-0827’ (unpatented) and the male parent, ‘OE-2000-BA-01’ (unpatented), as described in Table 1.

TABLE 1
Comparison with Parental Lines

| | | Female Parent | Male Parent |
|---|---|---|---|
| Characteristic | ‘KLEOE14243’ | ‘OE-2009-0827’ | ‘OE-2000-BA01’ |
| Ray floret | Yellow | Yellow with pink | Yellow |
| color | | ring at the base | |
| Peduncle | Moderately | Moderately | Very long |
| length | long | long | |

‘KLEOE14243’ is most similar to the commercial Osteospermum variety ‘KLEOE11187’ (U.S. Plant Pat. No. 24,034). Differences between the varieties are described in Table 2.

TABLE 2
Comparison with Similar Variety

| Characteristic | ‘KLEOE14243’ | ‘KLEOE11187’ |
|---|---|---|
| Ray floret, | Absent to very | Medium |
| width of ring at the base | narrow | |
| Ray floret, | Obtuse | Acute |
| shape of apex | | |

We claim:

1. A new and distinct variety of Osteospermum plant designated ‘KLEOE14243’ as illustrated and described herein.
